(19) United States
(12) Patent Application Publication
(10) Pub. No.: US 2002/0169584 A1
(43) Pub. Date: Nov. 14, 2002

(54) Mobile monitoring system

(76) Inventors: Zhongsu Fu, Wakefield, MA (US); Haiping Luo, Malden, MA (US)

Correspondence Address: STAAS & HALSEY LLP, 700 11TH STREET, NW, SUITE 500, WASHINGTON, DC, 20001, US

(21) Appl. No.: 09/853,873

(22) Filed: May 14, 2001

(51) Int. Cl. G06F 11/00

(52) U.S. Cl. 702/188

(57) ABSTRACT

A system and method for a mobile monitoring system, comprising monitoring an entity with a monitor, recording information based on the monitoring, communicating said information to a wireless communication device when an emergency situation is detected based on the monitoring, forwarding said information from said wireless communication device to a server, determining an action to take based upon said information, and taking said action.

### BACKGROUND OF THE INVENTION

1. Field of the Invention

The present invention relates to monitoring systems. More specifically, the invention relates to a mobile monitoring system that monitors the health of individuals, recognizes anomalies based on the monitoring, diagnoses the cause of the anomalies, and takes corrective action based on the diagnosis. Additionally, the invention is able to monitor the state of electrical and/or mechanical devices, recognize anomalous states of the devices based on the monitoring, diagnose the cause of the anomalous states, and take corrective action based on the diagnosis.

2. Description of the Related Art

The ability to determine whether individuals out of reach of immediate medical assistance, such as hospital out-patients or military personnel deployed in hostile territory, are in need of medical assistance has been a top priority for both hospitals and militaries alike. To support this priority, the prior art has attempted to produce effective medical monitoring systems to remotely monitor the well-being of patients and personnel. Of these attempts, the prior art generally consists of two types of medical monitoring systems.

The first type of medical monitoring system typically includes a monitoring unit, affixed to a patient, that is in constant communication with a base station. In this type of monitoring system, the base station must be in close proximity to the patient. This is due to the short range communication system used between the monitoring unit and base station—which creates a small communication zone within which the patient must remain.

Problems exist with this first type of medical monitoring system. One problem is limited mobility, as the patient must remain within the communication zone or else the patient will no longer be monitored by the base station. Furthermore, the constant communication between the monitor and base station causes continuous communication traffic and interference and a continuous drain of the monitoring unit's power supply, whether or not the patient has a medical problem that needs to be analyzed or interpreted by the base station.

The second type of monitoring system typically incorporates a mobile phone into the monitor itself, which is worn by the patient being monitored, and which is in communication with a central monitoring device via a wireless communication network.

There are several disadvantages of using the second type of monitoring system, the first of which is wasted/uneven power consumption due to the combination cell phone/monitor relying on the same power supply. This means, for instance, that the single power supply may be unduly drained by cell phone calls, leaving little power remaining for the actual monitoring of the patient.

Another disadvantage of the prior art is that the communication between the monitor and server is a one-way only continuous communication. This continuous communication results in a waste of resources in the communication network. Moreover, the cell phone inside the monitor is restricted in use for monitoring purposes, and may not be used for any other purpose.

Therefore, a need exists for a base station independent, unlimited mobility mobile monitoring system with reduced power consumption, reduced network utilization, and a plurality of accessible communication channels.

### SUMMARY OF THE INVENTION

An aspect of the present invention is to provide a mobile monitoring system that monitors the health of individuals while allowing the monitored individuals to travel anywhere in the world at any time.

Another aspect of the present invention is to provide a mobile monitoring system that monitors the health of patients with a monitor and is able to make a decision whether to call 911 and/or notify doctors based on the collected data from the monitor and the patient's profile.

A further aspect of the present invention is to monitor the health of an individual with a monitor and make the location of the individual along with the collected health information from the monitor immediately available to an emergency service personnel in an auditory and/or electronic format.

Yet another aspect of the present invention is to monitor the vital signs of an individual with a monitor, recognize anomalous vital sign readings, and instruct the monitor and/or individual how to correct the anomalous readings.

Another aspect of the present invention is to provide a mobile monitoring system that is able to monitor, diagnose, and correct problems in electrical and/or mechanical devices.

The above aspects may be attained by a system and method that monitors an entity with a monitor, records information based on the monitoring, communicates the information to a wireless communication device, forwards the information from the wireless communication device to a server, determines an action to take based upon said information, and takes the determined action.

The above aspects may also be attained by a system and method that gathers entity information from an entity with a monitor, determines anomalous entity readings from the gathered entity information, determines curative instructions to correct a cause of the anomalous entity readings, and sends the curative instructions to the monitor,

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 depicts a mobile patient/individual monitoring system according to an embodiment of the present invention.

FIG. 2 depicts a mobile mechanical/electrical device monitoring system according to an embodiment of the present invention.

FIG. 3 provides a more detailed view of system elements depicted in FIGS. 1 and 2.

FIG. 4 depicts a reference implementation of server 250 depicted in FIG. 3 according to an embodiment of the present invention.

FIG. 5 depicts a mobile monitoring system flow according to an embodiment of the present invention.

### DESCRIPTION OF THE PREFERRED EMBODIMENTS

Reference will now be made in detail to the preferred embodiments of the present invention, examples of which are illustrated in the accompanying drawings, wherein like reference numerals refer to like elements throughout.

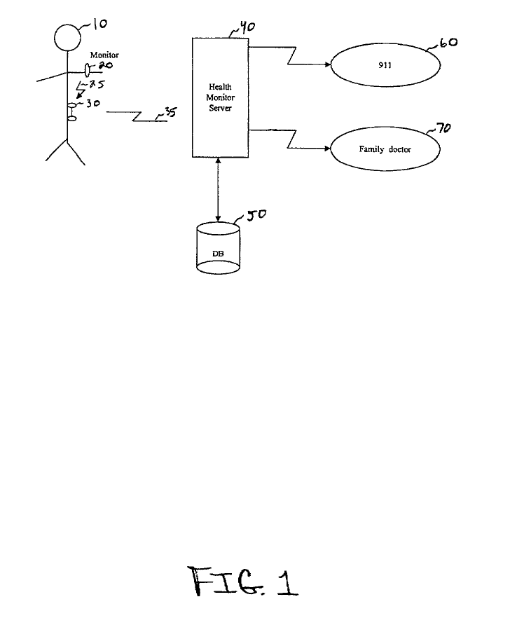

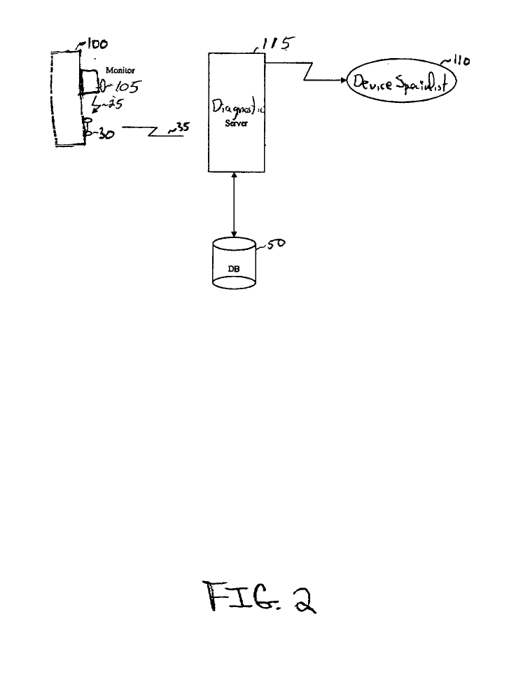

FIG. 1 depicts a mobile patient/individual monitoring system according to an embodiment of the present invention. The mobile patient/individual monitoring system provides a unique solution to patients who are in need constant health monitoring, and the system automatically calls for medical help for such patients in the event that they suffer from a detected critical medical condition or other type of detected medical condition.

Medical monitor 20, wearable by patient 10, is used to monitor the vital signs of patient 10. Medical monitor 20 is capable of monitoring a multitude of medical parameters, including but not limited to heart rate, temperature, and blood pressure. Medical monitor 20 stores monitored data over time and detects abnormalities in the monitored data gathered from patient 10. Medical monitor 20 works in cooperation with communication device 30 which may be a personal digital assistant (PDA), cell phone, computer with a modem communicating through a landline phone, or any other type of communication device. Medical monitor 20 communicates with communication device 30 via communication link 25 using wireless Bluetooth technology or any other type of communication technology. Advantageously, medical monitor 20 only communicates with communication device 30 when monitor 20 detects an anomaly in the monitor readings of patient 10, thereby reducing network traffic and conserving the battery life of monitor 20 and communication device 30.

After receiving a signal containing monitoring data from medical monitor 20 via communication link 25, communication device 30 determines the location of patient 10, using, for example, a location server, an attached GPS locator module, or any other type of individual tracking device. Communication device 30 next establishes a connection with health monitor server 40 via communication link 35. The connection between communication device 30 and health monitor server 40 may be a wireless or wired connection over the Internet utilizing TCP/IP, or over any other type of network.

Health monitor server 40 analyzes the data gathered by medical monitor 20 in conjunction with a patient profile of patent 10, which is stored in database 50. Health monitor server 40 determines if patient 10 has certain specified conditions, including but not limited to a heart attack or dangerous blood pressure levels. If health monitor server 40 determines that the monitoring data indicates that patient 10 requires immediate medical attention, then health monitor server 40 contacts appropriate emergency medical personnel 60 by, for example, calling 911 and informing 911 of the location of patient 10 using a pre-recorded message, voice synthesis, e-mail, and/or an SMS message. In addition, the collected data from patient 10 and/or an initial diagnostic decision made by health monitor server 40 may also be conveyed to 911 through voice synthesis, fax, e-mail, or by any other electronic means, so that emergency service personnel 60 may be better prepared to treat patient 10. If, on the other hand, health monitor server 40 determines that the monitoring data does not indicate an emergency, but instead determines that the medical doctor or family doctor of patient 10 should be contacted for any reason, then health monitor server 40 contacts doctor 70 and informs doctor 70 about the current condition of patient 10 using, for example, text to speech voice synthesis, e-mail, and/or an SMS message. Moreover, the doctor may request and receive additional patient information from health monitor server 40. Health monitor server 40 may receive the doctor's request by utilizing voice-recognition technology, or it may, for example, parse an e-mail or SMS message sent in by the doctor. Doctor 70 and emergency medical personnel 60 may also request and retrieve information from health monitor server 40 via the Internet.

Alternatively, in the event of a non-emergency, health monitor server 40 may send recommendations, such as a proposed diet, to patient 10 based upon an analysis of the medical monitoring data via communication link 35 and communication device 30.

According to another embodiment of the present invention, patient 10 wears several monitor devices in communication with each other to form an Ad-hoc network, using, for example, Bluetooth technology to form said network, although any other type of networking technology may be employed. This network of monitors communicates with health monitor server 40 via communication device 30.

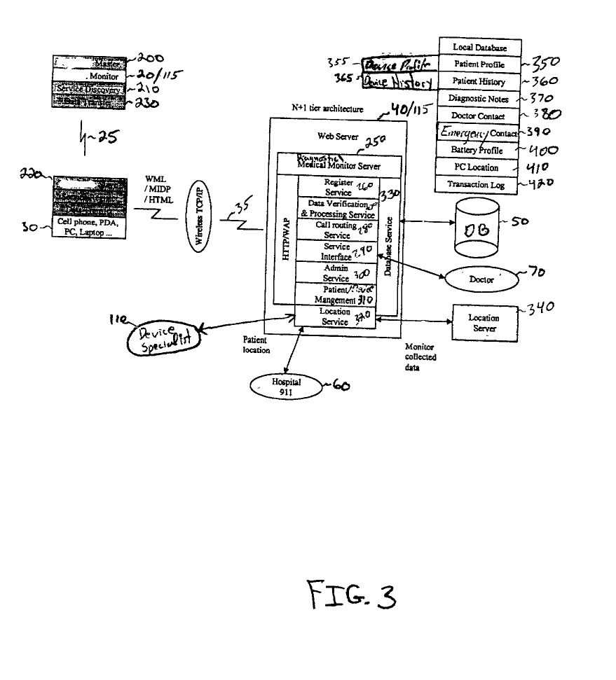

FIG. 2 depicts a mobile mechanical/electrical device monitoring system according to an embodiment of the present invention. Device monitor 105, incorporating a data storage means, is affixed to device 100. It is to be understood that monitor 105 may be a single monitor, or multiple monitors networked together, affixed to or located approximate to device 100. Device 100 may be a mechanical device, an electrical device, or any other type of device which has parameters which may be monitored for correctness. Device monitor 105 may be battery powered, or, alternately, it may draw power from device 100. Device monitor 105 monitors and the status of device 100. Device monitor 105 includes a user-configurable threshold alarm setting that determines when device 100 is malfunctioning, and wireless communications with an associated communication channel for communicating the malfunction to communication device 30 via communication link 25. Communication device 30 may be a personal digital assistant (PDA), cell phone, computer with a modem communicating through a landline phone, or any other type of communication device. Communication device 30 may be affixed to device 100, or, alternately, it may be situated apart from device 100. When communication device 30 receives monitoring data from device monitor 105, communication device 30 transmits the data received to diagnostic server 115 via communication link 35 where it is processed and stored. Moreover, after receiving a signal containing monitoring data from device monitor 105 via communication link 25, communication device 30 determines the location of device 100, using, for example, a GPS locator module affixed to device 100, or any other type of tracking device.

Communication link 35 may be a radio connection to a base station (not shown) which accesses standard communication channels to convey the collected monitoring data from device 100 to diagnostic server 115. Alternatively, communication link 35 may be a direct radio or landline connection which transmits the monitoring data directly to diagnostic server 115.

Diagnostic server 115 analyzes the data gathered by device monitor/monitors 105 in conjunction with a device profile of device 100, which is stored in database 50. Diagnostic server 115 determines if device 100 is outside the range of normal operating parameters. If diagnostic server 115 determines that the monitoring data indicates that device 100 requires immediate attention, then diagnostic server 115 contacts appropriate emergency device specialists 110 by, for example, automatically calling device specialist 110 and informing device specialist 110 of the location and problems with device 100 using a pre-recorded message and/or voice synthesis. In addition, the collected data from device 100 and/or an initial diagnostic decision made by diagnostic server 115 may also be conveyed to device specialist 110 through voice synthesis, fax, e-mail, or by any other electronic means, so that device specialist 110 may be better able to fix device 100.

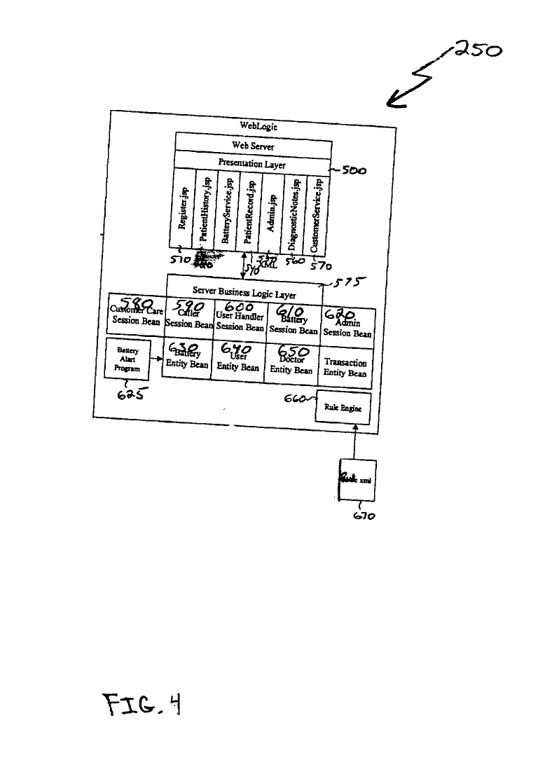

FIG. 3 provides a more detailed view of system elements depicted in FIGS. 1 and 2. Master 200 is used to register patient 10 or device 100. When a patient or device owner purchases monitor 20 or 105, respectively, a registration service may be used to register the monitor. In the event of the system of FIG. 1, a patient profile record is generated in database 50 and the monitor life start date time and the battery life span are also recorded in database 50. In the event of the system of FIG. 2, a device profile record is generated in database 50 and the monitor life start date time and the battery life span are also recorded in database 50.

A monitor may also be de-registered when a patient or device owner discontinues the monitoring service. The patient/device profile and history may then be deleted, or achieved for later retrieval.

Service discovery 210 automatically finds a slave device, such as slave 220, which previously registered with master 200. Master 200 checks if slave 220 has the capability to transfer data to a server such as health monitor server 40 or diagnostic server 115. If such a slave device can not be found or is in incapable of transferring data, then a beep or a vibration signal is triggered to notify patient 10 or a device 100 user to turn on or find such a slave device.

Monitor 20 detects, collects, and stores the medical data from patient 10 such as time and heart beat rate, blood pressure, temperature, psychological, and environment conditions. Monitor 115 detects, collects, and stores diagnostic data from device 100 including but not limited to internal/external operating temperature, internal pressure, voltage levels, input/output data, and/or environmental conditions. Monitors 20 and 115 store data in an internal memory and make decisions as to when the condition of the entity being monitored becomes critical. At this critical time, master 200 triggers a program on slave 220 using data transfer 230 that passes the data to health monitor server 40 in the event that a patient or individual is being monitored as in FIG. 1, or to diagnostic server 115 in the event that a device is being monitored as in FIG. 2.

Data transfer 230 organizes the monitoring data in a format that is suitable for the selected slave device. The data format depends on whether communication device 30 is a PDA, a cell phone, a laptop, PC, or other type of communication device.

In the event that communication device 30 is a wireless phone and is currently being used or is in voice mode, then monitors 20 and 115 are able to interrupt the call and switch slave 220 to data mode, as is known in the art (see, for example. U.S. Pat. No. 5,815,503), so that the monitoring data may be sent out immediately.

In the event that communication device 30 is a landline phone, there are at least two methods for transferring data through a land phone. One method utilizes a modem installed in a computer, where the computer has a wireless communication with the monitor through the modem. In this case, the computer is enabled to interrupt the voice call and do autodialing for the user, by using, for example, TAPI software.

Another method for transferring data through a landline phone is used when there is no computer present. When there is no computer involved, there is typically a wireless communication channel open between the phone and the monitor. In this situation, software controls the communication channel open between the phone and monitor, and can interrupt the land phone. Alternately, a multi-channel modem may be used to communicate with a backend server using a data channel to transfer data. In an alternative embodiment, a voice board may be used to provide s/w capability to detect an inbound signal, and to switch the voice channel to a data channel when such an inbound signal is detected. In yet another embodiment, a multi-channel modem may be used to simultaneously transmit voice and data communications to a remote site (see, for example, U.S. Pat. Nos. 5,452,289 and 5,535,204).

Web servers 40 and 115 may be any computer servers, such as Apache, IIS, or Enterprise Server, used as a front end for diagnostic/medical monitor server 250. Web servers 40 and 115 accept HTTP requests from cell phones, PDAs, PCs, laptops, or any Bluetooth or other enabled device that communicates with a registered monitor, such as monitors 20 and 105. Alternately, web servers 40 and 115 may be a part of middleware including but not limited to WebLogic.

The different components of diagnostic/medical monitor server 250 will now be described. Register Service 260 creates and stores patient and device profiles and other relevant data into database 50. Data Verification and Data Processing Service 270 verifies incoming data and analyses the collected data and makes a decision on what to do. The decision is based on a set of rules built into server 250. For instance, if the data indicates an emergency, server 250 calls 911 for the patient along with the location of the patient. If data indicates that the patient needs to take a certain action by him/herself then server 20 sends an alarm message to the patient along with medical or curative instructions. Each individual's case and medical domain knowledge may be used in determining these rules. Call Routing Service 280 makes calls, sends data or formatted graphs to a determined location, such as emergency rooms, doctors, or device specialists. Service Interface 290 provides a gateway to external systems such as legacy systems and preparatory systems. Database Service 330 provides utilities to insert, delete and modify data in database 50. Admin Service 300 provides a user interface for the administrator system status. Patient/Device management 310 includes tools and utilities to allow customer service to update customer records. Location Service 320 finds the location of a patient or device at calling time. There are multiple methods to find the location which depends upon the device used. For instance, if the call is from a cell phone or PDA then location server 340 will provide the location to location service 320, or if the call is from a PC then location service 320 service will look up the registered location of the PC.

The different data fields of database 50 will now be described. Patient Profile 350 the patient profile which is created when a customer or patient first uses a monitor. Patient Profile 350 contains such data fields as name of patient or individual, phone number, address, and the conditions to be monitored. Device Profile 355 contains such data fields as name, phone number, address, and the systems/parameters of device 100 to be monitored. Patient History 360 is the data collected from monitoring the patient 10 or other individuals. Patient History 360 contains, but is not limited to containing, such information as heart beat rate and the date and time that the data is collected. Device History 365 is the data collected from monitoring device 100. Diagnostic Notes 370 contains a doctor's or device specialist's comments on each patient or device. Doctor Contact 380 contains the family doctor's name, address, telephone number and data transfer SPI details of each patient 10. Emergency contact 390 stores the emergency phone numbers of emergency contacts such as doctors 70, emergency medical personnel 60, or device specialists 110. Monitor Battery Profile 400 contains identification number of monitor 20 and the battery expired date. PC Location 410 stores the location of the PC, if a PC is being used as communication device 30. Transaction Log 420 stores each connection from communication device 30.

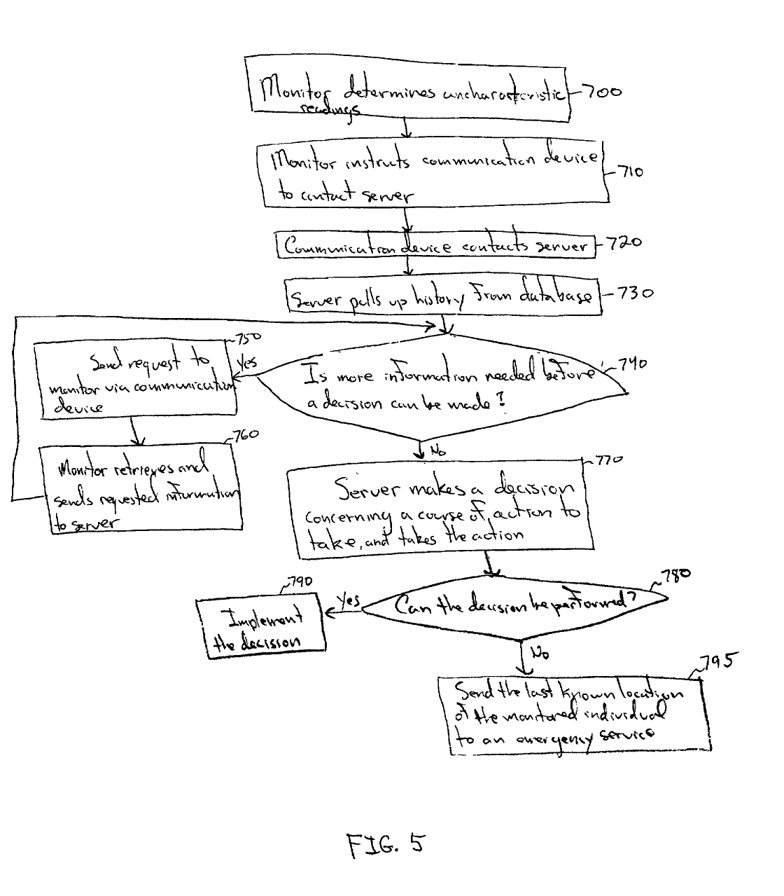

FIG. 4 depicts a reference implementation of server 250 depicted in FIG. 3 according to an embodiment of the present invention. This particular embodiment is based on J2EE and WebLogic. However, it is to be understood that there are alternative approaches and embodiments for the implementation of server 250 including, but not limited to, ASP and CGI.

Presentation layer 500 provides a GUI interface to different users, such as administrators and service people, to perform a set of tasks. Register.jsp 510 is a GUI interface which allows users to enter registration information. Register.jsp 510 communicates with User Handler Session Bean 600 to store registration information into database 50. PatientHistory.jsp 520 is a GUI interface for allowing a user to view patient/device history information. PatientHistory.jsp 520 communicates with User Handler Session Bean 600 to store and retrieve patient history information from patient history 360. BatteryService.jsp 530 is a GUI that allows users to insert and update monitor battery information such as the life expectancy and expire date of the battery or batteries used in monitor 20 and monitor 105. PatientRecord.jsp 540 is a GUI which allows users to view patient records. PatientRecord.jsp 540 communicates with User Handler Session Bean 600 to retrieve data from patient history 360. Admin.jsp 550 is a utility to allow an administrative user to maintain and trouble-shoot diagnostic/medical monitor server 250. Admin.jsp 550 communicates with Admin Session Bean 620 to store and retrieve information from transaction log 420. DiagnosticNote.jsp 560 is a GUI that allows a doctor or device specialist to insert and update diagnostic information stored in database 50 through Doctor Entity Bean 650. CustomerService.jsp 570 is a GUI which allows a user to solve problems encountered by customers by communicating with Customer Care Session Bean 580. Business Logic Layer 575 provides business logic related to the health/diagnostic service. Caller Session Bean 590 calls User Handler Session Bean 600 to write data into transaction log 420 for a particular call from communication device 30. User Handler Session Bean 600 creates and writes data in patient profile 350, device profile 355, doctor contact 380, and battery profile 400 using User Entity Bean 640 and Doctor Entity Bean 650. Battery Session Bean 610 calls Battery Entity Bean 630 to update battery profile 400.

Rule Engine 660 at system start time, or dynamically upon command, loads and parses rule file 670. Rule Engine 600 then translates rule file 670 into algorithms for use by User Handler Session Bean 600 and Doctor Entity Bean 650. Rule file 670 stores all of the rules for the decision algorithms. Rule file 670 is user-configurable. Typically, but not necessarily, doctors, medical experts, and/or device specialists generate the rules stored within rule file 670.

Battery Alert Program 625 provides alert signals to communication device 30.

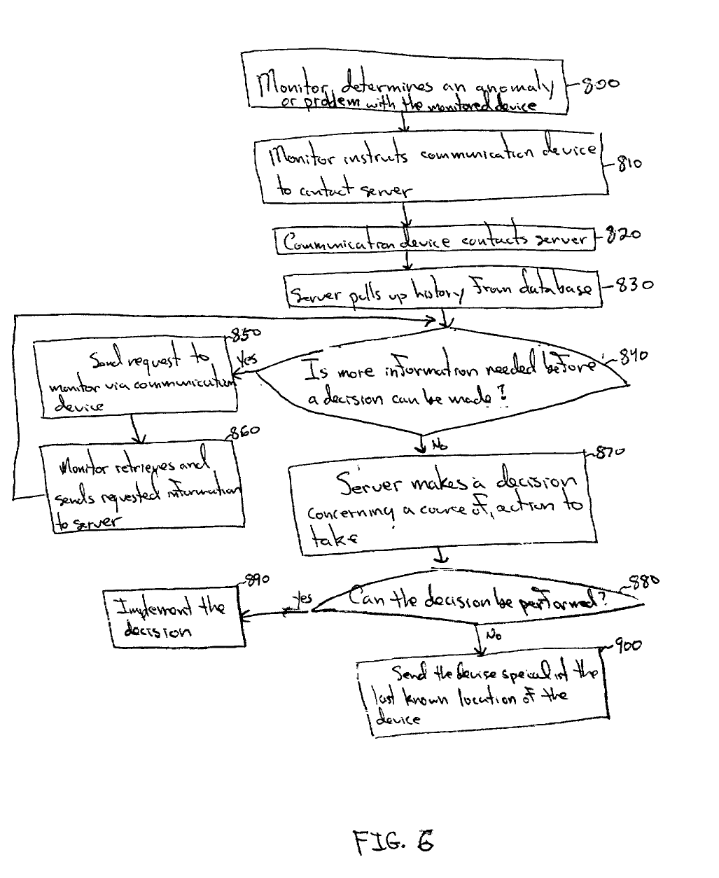

FIG. 5 depicts a mobile monitoring system flow according to an embodiment of the present invention. At operation 700, monitor 20 carried by patient 10 determines that one or more anomalies or health problems exist in patient 10 based upon monitoring data generated by the monitoring of patient 10. At operation 710, monitor 20 instructs communication device 30 to communicate with health monitor server 40 and forwards the monitoring data to communication device 30. A operation 720, communication device 30 contacts health monitor server 40 and sends the monitoring data to health monitor server 40. At operation 730, health monitor server retrieves the patient profile and patient history of patient 10, runs an analysis of the monitoring data, and, at operation 740, determines if more monitoring information is needed from monitor 20 before a decision as to a course of action may be made. If more monitoring information is needed, then health monitor server 40 sends a request to monitor 20 via communication device 30. At operation 760, monitor 20 receives the server's request and sends the requested information to health monitor server 40 via communication device 30, and processing loops back to operation 740.

If, on the other hand, more monitoring information is not needed, then processing proceeds to operation 770 and health monitor server 40 makes a decision as to a course of action to take, such as contacting a doctor, calling 911, sending instructions to patient 10, or sending instruction to monitor 20 to, for instance, increase the dosage of a drug to patient 10. This decision is based upon the monitoring data, the patient's profile and history, and any additional monitoring data received by monitor 20 if operations 750 and 760 are implemented.

At operation 780, health monitor server 40 determines if the determined action can be taken. If so, then processing proceeds to operation 790 where the decision is implemented. If, on the other hand, the decision can not be performed for any reason, including communication problems, then processing proceeds to operation 795 at which point the last known location of patient 10 is sent to emergency medical personnel 60 so that patient 10 may be found and treated by said emergency medical personnel as quickly as possible.

FIG. 6 depicts a mobile monitoring system flow according to an embodiment of the present invention. At operation 800, monitor 105 affixed to device 100 determines that one or more anomalies or problems exist in device 100 based upon monitoring data generated by the monitoring of device 100. At operation 810, monitor 105 instructs communication device 30 to communicate with diagnostic server 115 and forwards the monitoring data to communication device 30. A operation 820, communication device 30 contacts diagnostic server 115 and sends the monitoring data to diagnostic server 115. At operation 830, server 115 retrieves the device profile and device history of device 100, runs an analysis of the monitoring data, and, at operation 840, determines if more monitoring information is needed from monitor 105 before a decision as to a course of action may be made. If more monitoring information is needed, then diagnostic server 115 sends a request to monitor 105 via communication device 30. At operation 860, monitor 105 receives the server's request and sends the requested information to diagnostic server 115 via communication device 30, and processing then loops back to operation 840.

If, on the other hand, more monitoring information is not needed, then processing proceeds to operation 870, and diagnostic server 115 makes a decision as to a course of action to take, such as contacting a diagnostic specialist for instructions, or instructing monitor 105 to repair device 100 by, for example, sending electronic pulses and/or providing needed fluid to specific parts of device 100. This decision is based upon the monitoring data, the device's profile and history, and any additional monitoring data received by monitor 105 if operations 850 and 860 are implemented.

At operation 880, diagnostic server 115 determines if the determined action can be taken. If so, then processing proceeds to operation 890 where the decision is implemented. If, on the other hand, the decision can not be performed for any reason, such as a communication problems, then processing proceeds to operation 900 at which point the last known location of device 100 is sent to an emergency service number so that a device specialist may be contacted to find device 100 and make any necessary repairs.

Although preferred embodiments of the present invention have been shown and described, it will be appreciated by those skilled in the art that changes may be made in these embodiments without departing from the principle and spirit of the invention, the scope of which is defined in the appended claims and their equivalents.

What is claimed is:

1. A system, comprising:
a monitor for monitoring an entity;
a server for storing entity information; and
a communication device situated apart from said monitor for communicating with said monitor and/or server.

2. The system of claim 1, wherein the entity is a patient, and the entity information comprises a patient profile.

3. The system of claim 1, wherein the entity is a living being.

4. The system of claim 3, wherein the living being is a person.

5. The system of claim 1, wherein the entity is a mechanical device.

6. The system of claim 1, wherein the entity is an electrical device.

7. The system of claim 1, wherein communication device is a wireless phone.

8. The system of claim 1, wherein the communication device is a portable computer.

9. The system of claim 1, wherein the communication device is a PDA.

10. A method, comprising:
monitoring an entity with a monitor;
recording information based on the monitoring;
communicating said information to a wireless communication device;
forwarding said information from said wireless communication device to a server;
determining an action to take based upon said information; and
taking said action.

11. A method, comprising:
monitoring an entity with a monitor;
determining anomaly information based on the monitoring;
communicating said anomaly information to a wireless communication device;
forwarding said anomaly information from said wireless communication device to a computer;
retrieving entity information from a database;
determining an action to take based upon said anomaly information and/or said entity information; and
taking said action.

12. The method of claim 11, wherein said determined action comprises requesting additional information from said monitor.

13. The method of claim 11, wherein said determined action comprises forwarding said anomaly information and/or said entity information to a medical professional.

14. The method of claim 11, wherein said determined action comprises forwarding an emergency message and/or said entity information to an emergency medical service.

15. The method of claim 11, wherein said determined action comprises calling 911.

16. The method of claim 11, further comprising:
diagnosing a medical problem.

17. The method of claim 11, further comprising:
diagnosing a mechanical problem.

18. The method of claim 11, further comprising:
diagnosing an electrical problem.

19. The method of claim 13, further comprising:
determining location information for said entity; and
forwarding said location information to said medical professional.

20. The method of claim 14, further comprising:
determining location information for said entity; and
forwarding said location information to said emergency medical service.

21. The method of claim 11, wherein said entity is a patient and said entity information comprises a patient profile.

22. The method of claim 11, wherein said determined action comprises sending commands to said monitor.

23. The method of claim 11, wherein said determined action comprises sending instructions and/or an alarm message to said entity.

24. A method, comprising:
gathering entity information from an entity with a monitor;
determining anomalous entity readings from said gathered entity information;
determining curative instructions to correct a cause of said anomalous entity readings; and
sending said curative instructions to said monitor.

25. The method of claim 24, further comprising:
communicating said anomalous entity readings to a wireless communication device; and
forwarding said anomalous entity readings from said wireless communication device to a server.

26. The method of claim 24, wherein said curative instructions comprise instructing said monitor to increase a dosage of a medication.

27. The method of claim 24, wherein said curative instructions comprise instructing said monitor to send a electrical impulse to the entity.

28. The method of claim 24, wherein said curative instructions comprise increasing or decreasing the heart rate of said entity.
